(19) United States
(12) Patent Application Publication
(10) Pub. No.: US 2023/0200839 A1
(43) Pub. Date: Jun. 29, 2023

(54) TREATMENT TOOL AND METHOD OF ADJUSTING TREATMENT TOOL

(71) Applicant: Olympus Corporation, Tokyo (JP)

(72) Inventor: Seigo Kase, Sagamihara-shi (JP)

(21) Appl. No.: 18/171,011

(22) Filed: Feb. 17, 2023

Related U.S. Application Data
(63) Continuation of application No. PCT/JP2020/031902, filed on Aug. 24, 2020.

(51) Int. Cl. A61B 17/32; A61B 18/14

(52) U.S. Cl. CPC A61B 17/320092; A61B 18/1445; A61B 2017/320094; A61B 2017/2947

(57) ABSTRACT

A treatment tool includes: a first grasper; a second grasper that is rotatably provided with respect to the first grasper, the second grasper being configured to grasp a target region together with the first grasper by approaching the first grasper; a shaft configured to rotate the second grasper with respect to the first grasper; and an adjustment mechanism configured to adjust a trajectory along which the second grasper passes when the second grasper rotates about the shaft and approaches the first grasper, the adjustment mechanism including a protrusion provided according to a predicted length to be required due to variations in components.

### CROSS-REFERENCE TO RELATED APPLICATION

This application is a continuation of International Application No. PCT/JP2020/031902, filed on Aug. 24, 2020, the entire contents of which are incorporated herein by reference.

### BACKGROUND

### 1. Technical Field

The present disclosure relates to a treatment tool and a method of adjusting a treatment tool.

### 2. Related Art

As a medical treatment tool, a treatment tool that grasps a region (hereinafter, described as a target region) to be treated in a living tissue by a pair of graspers and performs treatment on the living tissue using ultrasonic vibration is known. For example, a treatment tool including a vibration transmission member that transmits ultrasonic vibration and a jaw provided rotatably with respect to the vibration transmission member is known (see, for example, WO 2017/047450 A). An operator such as a doctor grasps and holds a target region using a treatment tool, or applies ultrasonic vibration to the grasped target region to perform cauterization, coagulation, incision, and the like.

### SUMMARY

In some embodiments, a treatment tool includes: a first grasper; a second grasper that is rotatably provided with respect to the first grasper, the second grasper being configured to grasp a target region together with the first grasper by approaching the first grasper; a shaft configured to rotate the second grasper with respect to the first grasper; and an adjustment mechanism configured to adjust a trajectory along which the second grasper passes when the second grasper rotates about the shaft and approaches the first grasper, the adjustment mechanism including a protrusion provided according to a predicted length to be required due to variations in components.

In some embodiments, a treatment tool includes: a first grasper; a second grasper that is rotatably provided with respect to the first grasper, the second grasper being configured to grasp a target region together with the first grasper by approaching the first grasper; a shaft configured to rotate the second grasper with respect to the first grasper; a first gripped portion that is connected to a proximal end side of the first grasper; and a second gripped portion that is connected to a proximal end side of the second grasper, the treatment tool being configured such that a relative position between a first grasper and a second grasper is adjusted when the second grasper and the second gripped portion are rotatably coupled.

In some embodiments, provided is a method of adjusting a treatment tool. The method includes: checking a shift between a first grasper and a second grasper provided rotatably with respect to the first grasper in a state where the first grasper and the second grasper are aligned; and adjusting a relative position between the first grasper and the second grasper.

The above and other features, advantages and technical and industrial significance of this invention will be better understood by reading the following detailed description of presently preferred embodiments of the disclosure, when considered in connection with the accompanying drawings.

### BRIEF DESCRIPTION OF THE DRAWINGS

FIG. 1 is a view illustrating a treatment system according to a first embodiment of the disclosure;

FIG. 2 is a view illustrating a configuration of a main portion of the treatment tool illustrated in FIG. 1;

FIG. 3 is a cross-sectional view illustrating a configuration of the main portion of the treatment tool illustrated in FIG. 1;

FIG. 4 is a view (part 1) for describing an adjustment method according to the first embodiment of the disclosure;

FIG. 5 is a view (part 2) for describing the adjustment method according to the first embodiment of the disclosure;

FIG. 6 is a view illustrating a configuration of a main portion of a treatment tool according to a modification of the first embodiment of the disclosure;

FIG. 7 is a view (part 1) illustrating a configuration of a main portion of a treatment tool according to a second embodiment of the disclosure;

FIG. 8 is a view (part 2) illustrating a configuration of the main portion of the treatment tool according to the second embodiment of the disclosure;

FIG. 9 is a view (part 3) illustrating a configuration of the main portion of a treatment tool according to the second embodiment of the disclosure;

FIG. 10 is a view (part 4) illustrating a configuration of the main portion of the treatment tool according to the second embodiment of the disclosure;

FIG. 11 is a view for describing an adjustment method according to the second embodiment of the disclosure;

FIG. 12 is a view illustrating a configuration of a main portion of a treatment tool according to a modification of the second embodiment of the disclosure;

FIG. 13 is a view for describing an adjustment method according to the modification of the second embodiment of the disclosure;

FIG. 14 is a view illustrating a configuration of a main portion of a treatment tool according to a third embodiment of the disclosure;

FIG. 15 is a view (part 1) for describing an adjustment method according to the third embodiment of the disclosure;

FIG. 16 is a view (part 2) for describing the adjustment method according to the third embodiment of the disclosure;

FIG. 17 is a view (part 1) illustrating a configuration of a main portion of a treatment tool according to a fourth embodiment of the disclosure;

FIG. 18 is a view (part 2) illustrating a configuration of the main portion of the treatment tool according to the fourth embodiment of the disclosure;

FIG. 19 is a view for describing an adjustment method according to the fourth embodiment of the disclosure;

FIG. 20 is a view (part 1) illustrating a configuration of a main portion of a treatment tool according to another embodiment;

FIG. 21 is a view (part 1) illustrating a configuration of the main portion of the treatment tool according to another embodiment;

FIG. 22 is a cross-sectional view (part 1) illustrating a configuration of the main portion of the treatment tool according to another embodiment; and

FIG. 23 is a cross-sectional view (part 2) illustrating a configuration of the main portion of the treatment tool according to another embodiment.

### DETAILED DESCRIPTION

Hereinafter, an embodiment of a treatment tool according to the disclosure will be described with reference to the drawings. Note that the disclosure is not limited by the embodiments. In addition, in the description of the drawings, the same or corresponding elements are appropriately denoted by the same reference numerals. In addition, it should be noted that the drawings are schematic, and a dimensional relationship of each element, a ratio of each element, and the like may be different from reality. Portions having different dimensional relationships and ratios may be included between the drawings.

### First Embodiment

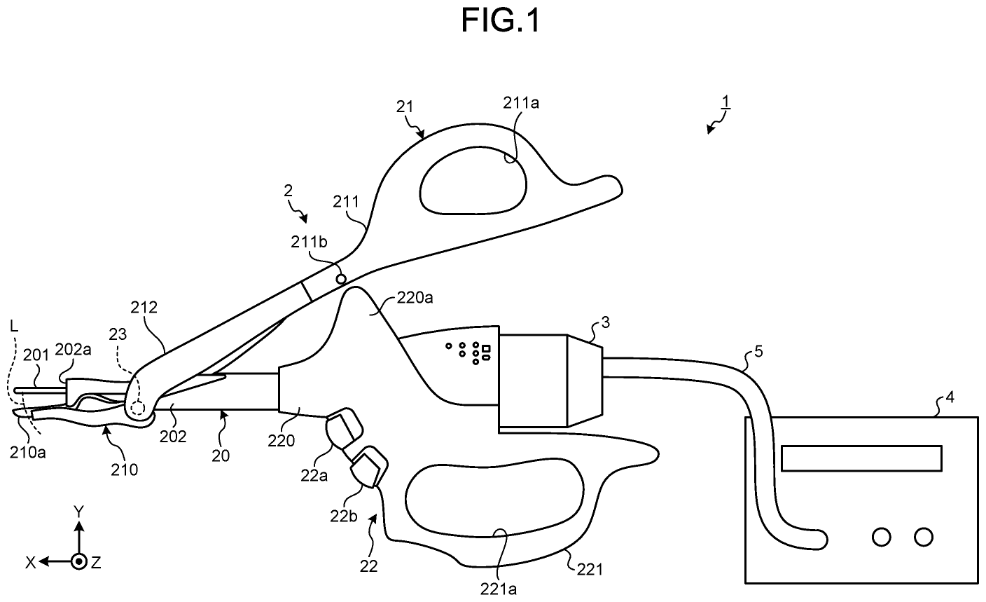

FIG. 1 is a view schematically illustrating a treatment system according to a first embodiment of the disclosure. A treatment system 1 is a system that performs cauterization, coagulation, incision, and the like by applying vibration or a high-frequency current to a grasped target region while grasping a region (target region) to be treated in a living tissue with a pair of grasper pieces. The treatment system 1 includes a treatment tool 2, a transducer unit 3, a control device 4, and a connection cable 5.

The treatment tool 2 applies heat to the grasped target region to perform cauterization, coagulation, incision, and the like on the target region. The configuration of the treatment tool 2 will be described later.

The transducer unit 3 generates ultrasonic vibration under the control of the control device 4. The transducer unit 3 includes, for example, an ultrasonic transducer. The ultrasonic transducer includes a piezoelectric element, and generates an ultrasonic wave by supplying a current to the piezoelectric element. The ultrasonic transducer is directly or indirectly connected to the treatment tool 2 (probe body 201 to be described later), and transmits the generated ultrasonic wave to the probe body 201. In addition, the transducer unit 3 is electrically connected to the control device 4 via the connection cable 5.

The control device 4 supplies electric power to the transducer unit 3 and the probe body 201 to control driving of the transducer unit 3. The control device 4 is configured using a general-purpose processor such as a central processing unit (CPU) or a dedicated processor such as various arithmetic circuits that execute specific functions such as a field programmable gate array (FPGA) and an application specific integrated circuit (ASIC).

The treatment tool 2 includes a probe portion 20, a first body portion 21, a second body portion 22, and a shaft 23.

The probe portion 20 includes a probe body 201 and a sheath 202.

The first body portion 21 includes a jaw 210, a gripped portion 211, and a cover 212.

The second body portion 22 includes a connection portion 220 and a gripped portion 221.

Note that, in the treatment tool 2, a side connected to the transducer unit 3 in the longitudinal direction of the sheath 202 is referred to as a “proximal end” side, and the opposite side is referred to as a “distal end” side. In addition, in three directions (X direction, Y direction, and Z direction) orthogonal to each other, the longitudinal direction of the sheath 202 of the treatment tool 2 is defined as the X direction, and the central axis direction of the shaft 23 is defined as the Z direction.

The probe body 201 is configured using a rod. The probe body 201 performs longitudinal vibration that vibrates in a direction parallel to the longitudinal direction (here, the X direction) of the probe body 201 by an ultrasonic wave transmitted from the transducer unit 3. By heat or friction generated by the longitudinal vibration, cauterization, coagulation, incision, and the like of the target region is performed. In addition, the probe body 201 is supplied with high-frequency power from the control device 4. When the high-frequency power is supplied, a high-frequency current is caused to flow to a target region by a potential difference generated between the probe body 201 and the jaw 210, and cauterization, coagulation, incision, and the like is performed. In a case where high-frequency power is supplied, the probe body 201 and the jaw 210 (grasp portion 210a) become electrodes through which high-frequency current flows.

In the first embodiment, the distal end of the probe body 201 corresponds to a first grasper.

The sheath 202, through which the probe body 201 is inserted, surrounds the probe body 201.

The jaw 210 is rotatably provided with respect to the probe portion 20. The jaws 210 penetrate the sheath 202 and rotate about an axis orthogonal to the longitudinal axis of the sheath 202. In addition, the jaw 210 grasps the target region together with the probe body 201 at one end. Specifically, the grasp portion 210a is provided at one end of the jaw 210. The jaw 210 swingably holds the grasp portion 210a. The grasp portion 210a rotates, for example, about an axis extending in a direction orthogonal to the longitudinal axis of the sheath 202. In addition, the jaw 210 is connected to the gripped portion 211 at the other end.

In the first embodiment, the distal end portion (grasp portion 210a) of the jaw 210 corresponds to the second grasper.

The gripped portion 211 is a portion gripped by the operator. A through hole 211a to be locked with a part (for example, a thumb) of the operator's hand is formed in the gripped portion 211.

The cover 212 covers a connection portion between the jaw 210 and the gripped portion 211.

The connection portion 220 holds the sheath 202 and is connected to the transducer unit 3. The connection portion 220 includes a guide 220a that sandwiches the gripped portion 211 and guides the rotation direction of the first body portion 21. The guide 220a has a concave shape facing each other with the gripped portion 211 sandwiched between when the gripped portion 211 moves toward the side of the second body portion 22.

The gripped portion 221 is a portion gripped by the operator. A through hole 221a to be locked with another portion (for example, an index finger or a middle finger) of the operator's hand is formed in the gripped portion 221.

The second body portion 22 is provided with operation buttons 22a and 22b. For example, the operation button 22a is a button for generating ultrasonic vibration in the probe body 201. In addition, the operation button 22b is a button for supplying high-frequency power to the probe body 201 to cause a high-frequency current to flow to a target region. Each button is pressed by the operator to output a signal to the control device 4. The control device 4 drives the transducer unit 3 or supplies high-frequency power to the probe body 201 according to the input signal.

The shaft 23 has a columnar shape and is provided to penetrate the sheath 202 and the jaw 210. Both end portions of the shaft 23 are held by the jaws 210. Therefore, the jaw 210 can slide with respect to the shaft 23 and is rotatable with respect to the sheath 202. Specifically, the shaft 23 penetrates the jaws 210 at both end portions, and rotatably holds the jaws 210 about the central axis. This central axis is the central axis of the shaft 23, extends in the Z direction of FIG. 1, and is orthogonal to the longitudinal axis of the sheath 202.

The treatment tool 2 can rotate the jaw 210 about the shaft 23 (central axis) with respect to the probe body 201 by operating the gripped portions 211 and 221. At this time, the grasp portion 210a moves along a trajectory L according to the rotation of the jaw 210 and approaches or abuts on the probe body 201. The trajectory L is a path through which a predetermined position of the jaw 210 (grasp portion 210a) passes when the gripped portion 211 approaches or moves away from the gripped portion 221.

When the operation buttons 22a and 22b are pressed after the target region is sandwiched between the probe body 201 and the grasp portion 210a by rotating the jaw 210, energy caused by ultrasonic waves or high-frequency power is supplied to the probe body 201 under the control of the control device 4. By supplying energy to the probe body 201, cauterization, coagulation, incision, and the like of the target region is performed.

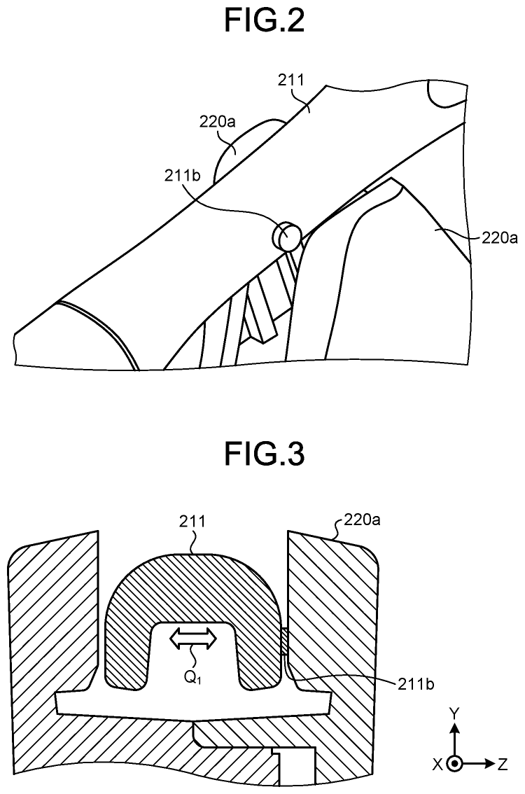

Next, an example of adjusting a position where the probe body 201 and the jaw 210 face each other in the treatment tool 2, particularly a relative position between the probe body 201 and the jaw 210 when a target region is grasped will be described with reference to FIGS. 2 and 3. FIG. 2 is a view illustrating a configuration of a main portion of the treatment tool illustrated in FIG. 1. FIG. 3 is a cross-sectional view illustrating a configuration of the main portion of the treatment tool illustrated in FIG. 1. FIG. 3 is a cross-sectional view illustrating configurations of the gripped portion 211 and the connection portion in a cross section taken along a plane passing through a protrusion 211b described later.

The position adjustment described below is performed, for example, in an inspection before shipping the treatment tool 2 in a factory where the treatment tool 2 is manufactured.

The connection portion 220 has a protrusion 211b that slides with respect to the guide 220a. The protrusion 211b protrudes from the body of the gripped portion 211. When the gripped portion 211 is accommodated in the guide 220a, the protrusion 211b comes into contact with the inner wall surface of the guide 220a. Thus, the position of the first body portion 21 with respect to the second body portion 22 is determined. In the first embodiment, the protrusion 211b corresponds to an adjustment mechanism.

The protrusion length of the protrusion 211b is set based on the relative position when the probe body 201 and the jaw 210 (grasp portion 210a) are brought close to each other. By adjusting the protrusion amount of the protrusion 211b, the position of the gripped portion 211 with respect to the connection portion 220 adjusted in the direction of an arrow Q. Here, the protrusion 211b is provided on the side opposite to the protrusion 211b with respect to the body of the gripped portion 211 depending on the direction in which the probe body 201 and the jaw 210 are shifted.

Note that the gripped portions 211 and 221 are formed by molding, for example. In addition, in a case where a required protrusion amount to be set due to variations in components and the like can be previously predicted, the protrusion 211b may be provided at the time of molding. In this case, for example, the mold for molding is configured to have a nested structure in a manner that the protrusion length can be adjusted to an arbitrary protrusion length.

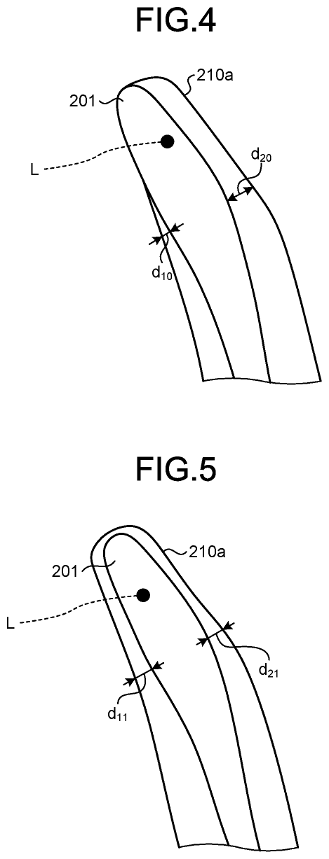

FIGS. 4 and 5 are views for describing an adjustment method according to the first embodiment of the disclosure. FIG. 4 is a view illustrating a state in which the grasp portion 210a is aligned with respect to the probe body 201, and illustrates positions of the probe body 201 and the jaw 210 (grasp portion 210a) in a case where the gripped portion 211 does not have the protrusion 211b. FIG. 5 is a view illustrating a state in which the grasp portion 210a is aligned with respect to the probe body 201, and illustrates positions of the probe body 201 and the jaw 210 (grasp portion 210a) in a case where the gripped portion 211 has the protrusion 211b.

In the state illustrated in FIG. 4, the position of the grasp portion 210a is shifted with respect to the probe body 201. Specifically, a distance d₁₀ between one outer edge of the probe body 201 and the outer edge of the grasp portion 210a is smaller than a distance d₂₀ between the other outer edge of the probe body 201 and the outer edge of the grasp portion 210a, and the relative position of the grasp portion 210a with respect to the probe body 201 is shifted. At this time, the trajectory L passing through the central portion of the grasp portion 210a is shifted with respect to the probe body 201. Note that the central portion here refers to a position of the center of gravity at a position where the grasp portion 210a originally faces the probe body 201.

On the other hand, in the state illustrated in FIG. 5, the grasp portion 210a is positioned at the center with respect to the probe body 201. Specifically, a distance d₁₁ between one outer edge of the probe body 201 and the outer edge of the grasp portion 210a is the same as a distance d₂₁ between the other outer edge of the probe body 201 and the outer edge of the grasp portion 210a. The position of the gripped portion 211 with respect to the guide 220a is adjusted by the protrusion 211b. Thus, the trajectory L when the grasp portion 210a approaches the probe body 201 is adjusted. When the trajectory L is adjusted, the relative positions of the probe body 201 and the grasp portion 210a when the probe body 201 and the grasp portion 210a approach each other are also adjusted. If the extended line of the trajectory L passes through the probe body 201, the target region can be sandwiched, but it is preferable that the trajectory L passes through the central portion of the probe body 201 from the viewpoint of securing characteristics such as incision and resistance.

In the first embodiment of the disclosure described above, the position of the first body portion 21 relative to the second body portion 22 is adjusted by providing the sliding protrusion 211b on the second body portion 22. According to the first embodiment, by adjusting the protrusion length of the protrusion 211b, it is possible to highly accurately adjust the position where the probe body 201 and the jaw 210 (grasp portion 210a), which are graspers for grasping the grasping target, face each other.

Note that, in the first embodiment, the side of the protrusion 211b attached to the gripped portion 211 may have a screw shape, and the amount of protrusion from the body of the gripped portion 211 may be adjusted by the amount of rotation of the protrusion 211b. By freely adjusting the protrusion amount of the protrusion 211b, the operator and the like can adjust the protrusion amount at the site of use.

In addition, in the first embodiment, a protrusion that abuts on a guide that is provided on the sheath 202 and guides the rotation trajectory of the jaw 210 may be provided on the distal end side of the jaw, and the probe body 201 and the grasp portion 210a may be adjusted in position by abutting.

Modification of First Embodiment

Next, a modification of the first embodiment will be described with reference to FIG. 6. FIG. 6 is a view illustrating a configuration of a main portion of a treatment tool according to a modification of the first embodiment of the disclosure. Note that the overall configuration of the treatment system according to the present modification is the same as that of the treatment system 1 described above except that the configuration of the protrusion is changed, and thus the description will be omitted. Hereinafter, a configuration different from that of the first embodiment will be described.

A protrusion 220b according to the modification is provided on the side of the guide 220a and abuts on the gripped portion 211. The protrusion 220b protrudes from the body of the connection portion 220. When the gripped portion 211 is accommodated in the guide 220a, the protrusion 220b comes into contact with the outer peripheral surface of the gripped portion 211. Thus, the position of the first body portion 21 with respect to the second body portion 22 is determined. The protrusion length of the protrusion 220b is set in the same manner as the protrusion 211b.

In the modification described above, the configuration of the protrusion is changed from that in the first embodiment described above, but the behavior of the treatment tool itself is not changed, in a manner that the same effect as that in the first embodiment can be obtained.

### Second Embodiment

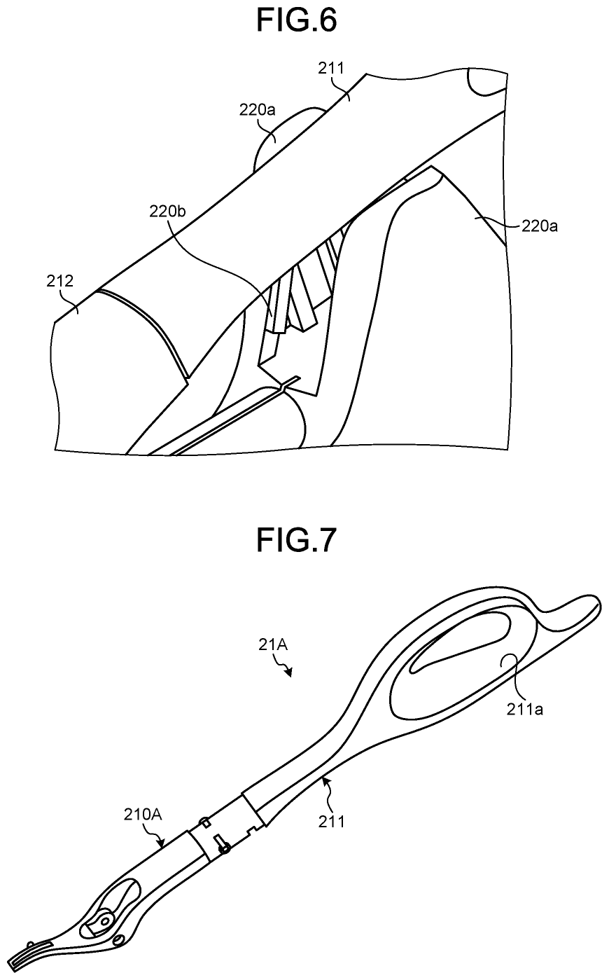

Next, a second embodiment will be described with reference to FIGS. 7 to 11. FIGS. 7 to 10 are views illustrating a configuration of a main portion of a treatment tool according to a second embodiment of the disclosure. Note that the overall configuration of the treatment system according to the second embodiment is the same as that of the treatment system 1 described above except that the adjustment mechanism of the relative position between the probe body and the jaw is changed, and thus the description will be omitted. Hereinafter, a configuration different from that of the first embodiment will be described.

The treatment tool according to the second embodiment includes a first body portion 21A instead of the first body portion 21 of the treatment tool 2 described above. A first body portion 21A includes a jaw 210A and the gripped portion 211. Note that, although the jaw 210A has the grasp portion 210a at the distal end, FIG. 7 and the like illustrate a configuration in which the grasp portion 210a is removed.

The jaw 210A rotates about the central axis of the shaft 23 and grasps the target region together with the probe body 201. The jaw 210A is connected to the gripped portion 211 at the other end.

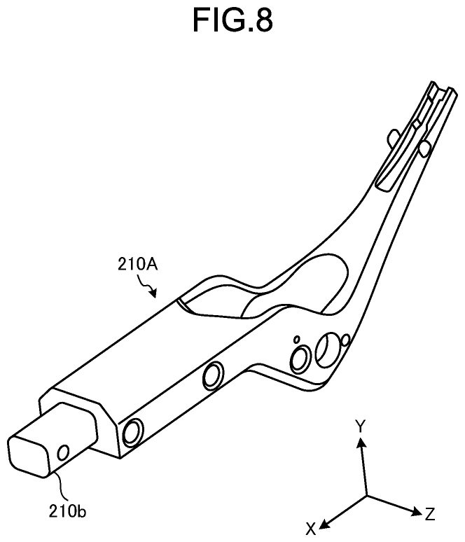

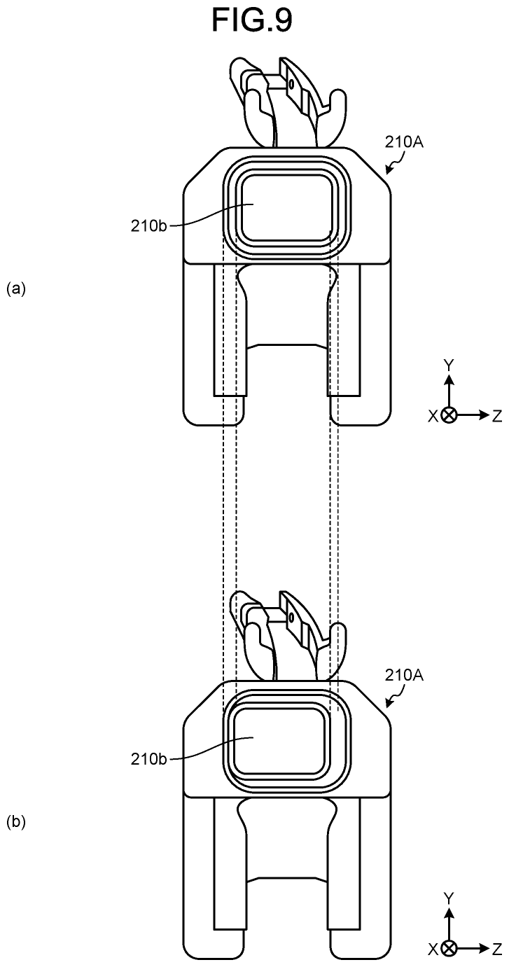

The jaw 210A has a convex portion 210b coupled to the gripped portion 211. The convex portion 210b extends in a direction parallel to the XY plane and is movable in the Z direction (see FIGS. 8 and 9). The position of the convex portion 210b is adjusted based on the positional relationship between the probe body 201 and the grasp portion 210a, and then fixed. For example, the convex portion 210b is adjusted from the initial position illustrated in (a) of FIG. 9 to the position illustrated in (b) of FIG. 9, and then fixed to the adjusted position.

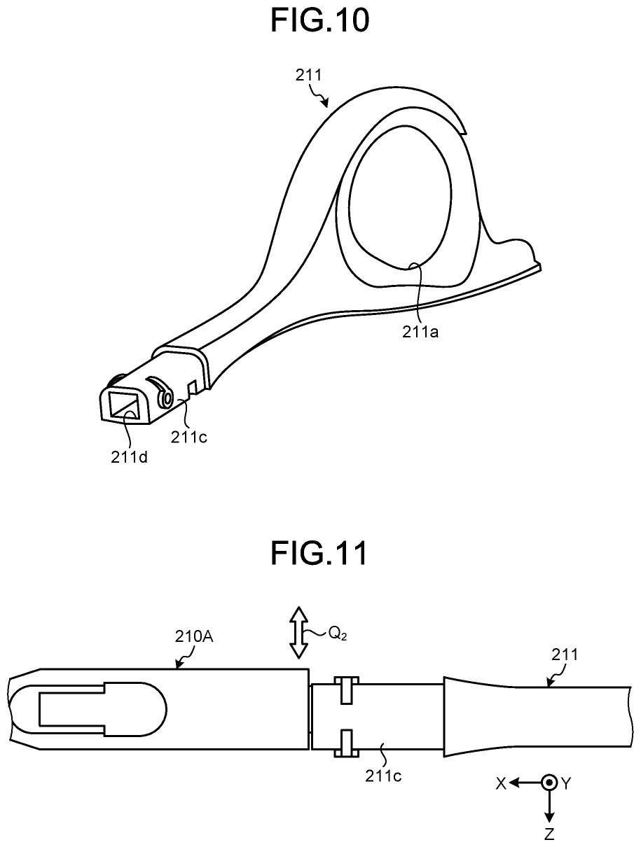

Here, the gripped portion 211 has a coupling portion 211c coupled to the jaw 210A. A hole 211d for accommodating the convex portion 210b of the jaw 210A is formed in the coupling portion 211c (see FIG. 10). Note that the coupling portion 211c is covered by the cover 212 at the time of assembling the treatment tool.

FIG. 11 is a view for describing an adjustment method according to the second embodiment of the disclosure. The convex portion 210b of the jaw 210A is inserted into the hole 211d and fixed. Thus, the jaw 210A is coupled to the gripped portion 211. At this time, the jaw 210A is adjusted in the direction of an arrow Q₂ with respect to the gripped portion 211 by the position of the convex portion 210b. By this adjustment, the trajectory L of the grasp portion 210a changes, and the relative position between the probe body 201 and the grasp portion 210a is adjusted. In the second embodiment, the adjustment mechanism is configured by the convex portion 210b and the hole 211d.

In the second embodiment described above, the configuration related to the position adjustment of the probe body 201 and the grasp portion 210a is changed from that in the first embodiment described above, but the behavior of the treatment tool itself after the adjustment is not changed, in a manner that the same effect as that in the first embodiment can be obtained.

Modification of Second Embodiment

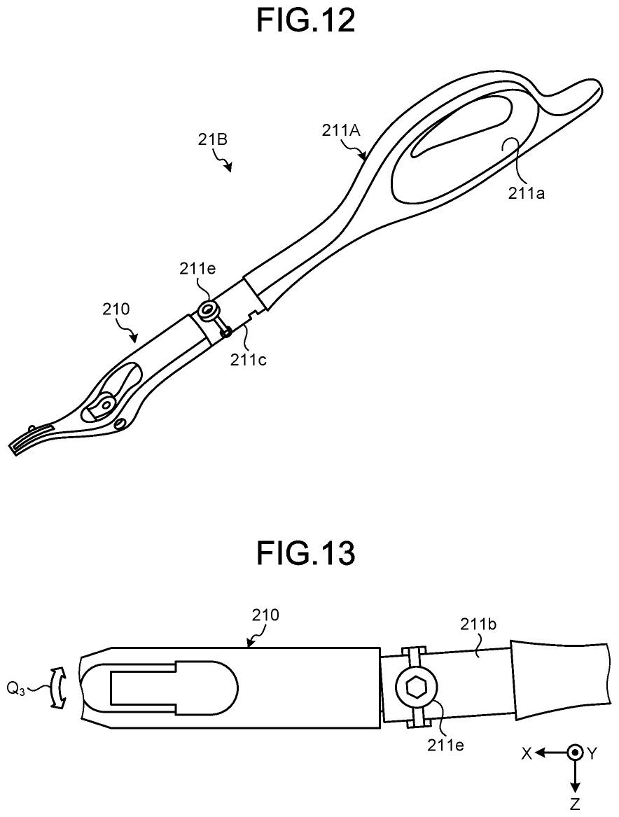

Next, a modification of the second embodiment will be described with reference to FIGS. 12 and 13. FIG. 12 is a view illustrating a configuration of a main portion of a treatment tool according to a modification of the second embodiment of the disclosure. Note that the overall configuration of the treatment system according to the present modification is the same as that of the treatment system 1 described above except that the adjustment mechanism of the relative position between the probe body and the jaw is changed, and thus the description will be omitted. Hereinafter, a configuration different from that of the first embodiment will be described.

The treatment tool according to the present modification includes a first body portion 21B instead of the first body portion 21 of the treatment tool 2 described above. The first body portion 21B includes the jaw 210 and a gripped portion 211A. Note that, although the jaw 210 has the grasp portion 210a at the distal end, FIG. 12 illustrates a configuration in which the grasp portion 210a is removed.

The gripped portion 211A is a portion gripped by the operator. The through hole 211a to be locked with a part (for example, a thumb) of the operator's hand is formed in the gripped portion 211A. In addition, the gripped portion 211A has the coupling portion 211c coupled to the jaw 210. Furthermore, the coupling portion 211c is provided with a rotation shaft 211e that rotatably couples the jaw 210. The rotation shaft 211e extends in the longitudinal direction of the gripped portion 211A and the direction orthogonal to the central axis of the shaft 23 (here, the Y direction).

The jaw 210 is supported by the rotation shaft 211e and coupled to the gripped portion 211. The jaw 210 is rotatable about the rotation shaft 211e. The rotation shaft 211e moves the jaw 210 on a plane intersecting the direction in which the probe body 201 and the jaw 210 face each other. Here, a direction in which the probe body 201 and the jaw 210 face each other is a direction parallel to the XY plane, and a plane intersecting with this direction is the XZ plane.

FIG. 13 is a view for describing an adjustment method according to the modification of the second embodiment of the disclosure. The position of the probe body 201 and the grasp portion 210a is adjusted by rotating the jaw 210 about the rotation shaft 211e. At this time, the jaw 210 is adjusted in the direction of an arrow Q₃ with respect to the gripped portion 211A. By this adjustment, the trajectory L of the grasp portion 210a changes, and the relative position between the probe body 201 and the grasp portion 210a is adjusted.

In the present modification described above, the configuration related to the position adjustment of the probe body 201 and the grasp portion 210a is changed from that in the second embodiment described above, but the behavior of the treatment tool itself after the adjustment is not changed, in a manner that the same effect as that in the second embodiment can be obtained.

### Third Embodiment

Next, a third embodiment will be described with reference to FIGS. 14 to 16. FIG. 14 is a view illustrating a configuration of a main portion of a treatment tool according to a third embodiment of the disclosure. Note that the overall configuration of the treatment system according to the third embodiment is the same as that of the treatment system 1 described above except that the adjustment mechanism of the relative position between the probe body and the jaw is changed, and thus the description will be omitted. Hereinafter, a configuration different from that of the first embodiment will be described.

The treatment tool according to the third embodiment includes a jaw 210B instead of the jaw 210 of the treatment tool 2 described above. The jaw 210B rotates about the central axis of the shaft 23 and grasps the target region together with the probe body 201. The jaw 210B is connected to the gripped portion 211 at the other end.

The jaw 210B has spacers 210c and 210d provided between the jaw 210B and the shaft 23. The spacer 210c is provided on one end side in the central axis direction of the shaft 23. The spacer 210d is provided on the other end side in the central axis direction of the shaft 23.

The spacers 210c and 210d have an annular shape (C-shape) which is formed by curving a belt-shaped member and is partially opened. Note that a protrusion may be provided on the inner peripheral side. The spacers 210c and 210d move the jaw 210B in the central axis direction of the shaft 23 by adjusting a length (hereinafter, referred to as “width”) of a portion corresponding to the central axis direction of the shaft 23. The arrangement of the spacers 210c and 210d adjusts the position of the jaw 210B with respect to the sheath 202. In the third embodiment, the spacers 210c and 210d correspond to an adjustment mechanism.

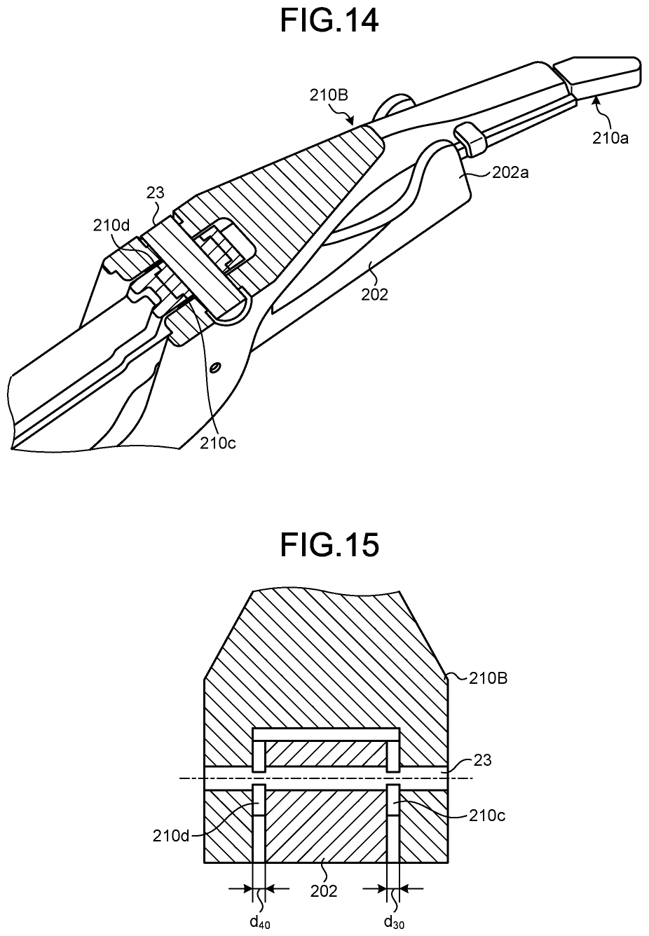

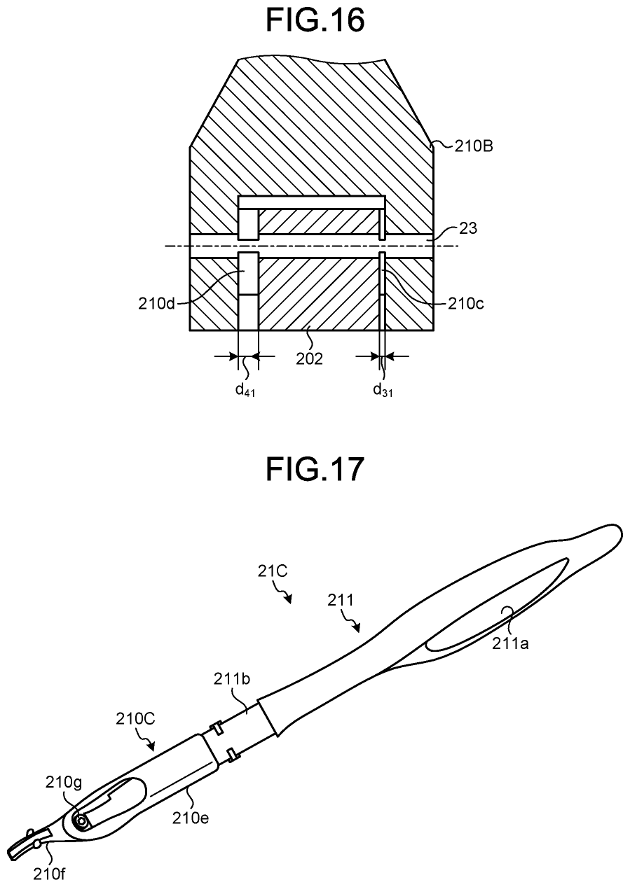

FIGS. 15 and 16 are views for describing an adjustment method according to the third embodiment of the disclosure. For example, in a case where the spacers 210c and 210d illustrated in FIG. 15 are provided, it is assumed that the probe body 201 and the grasp portion 210a are at the positions illustrated in FIG. 4. At this time, a width d₃₀ of the spacer 210c and a width d₄₀ of the spacer 210d are the same. At this time, for example, by adjusting the width of the spacer 210c to a width d₃₁ and adjusting the width of the spacer 210d to the width d₄₁ (>d₃₁), the trajectory L of the grasp portion 210a (the relative position between the probe body 201 and the grasp portion 210a) is adjusted to, for example, the positional relationship illustrated in FIG. 5.

In the third embodiment described above, the configuration related to the position adjustment of the probe body 201 and the grasp portion 210a is changed from that in the first embodiment described above, but the behavior of the treatment tool itself at the time of use is not changed, in a manner that the same effect as that in the first embodiment can be obtained.

### Fourth Embodiment

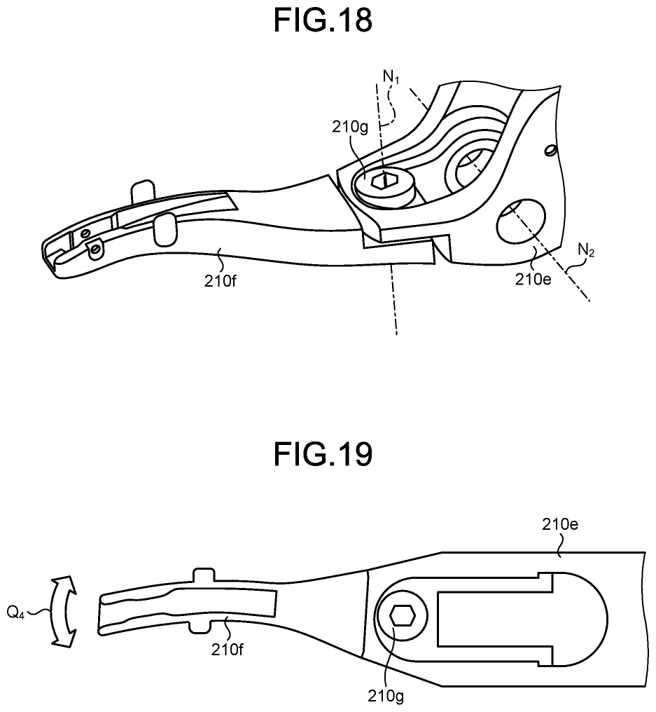

Next, a fourth embodiment will be described with reference to FIGS. 17 to 19. FIGS. 17 to 18 are views illustrating a configuration of a main portion of a treatment tool according to a fourth embodiment of the disclosure. Note that the overall configuration of the treatment system according to the fourth embodiment is the same as that of the treatment system 1 described above except that the adjustment mechanism of the relative position between the probe body and the jaw is changed, and thus the description will be omitted. Hereinafter, a configuration different from that of the first embodiment will be described.

The treatment tool according to the fourth embodiment includes a first body portion 21C instead of the first body portion 21 of the treatment tool 2 described above. A first body portion 21C includes a jaw 210C and the gripped portion 211. Note that, although the jaw 210C has the grasp portion 210a at the distal end, FIG. 17 and the like illustrate a configuration in which the grasp portion 210a is removed.

The jaw 210C rotates about the central axis of the shaft 23 and grasps the target region together with the probe body 201. The jaw 210C is connected to the gripped portion 211 at the other end.

The jaw 210C includes a first body portion 210e coupled to the gripped portion 211, a second body portion 210f that rotates with respect to the first body portion 210e, and a rotation shaft 210g that rotatably couples the second body portion 210f to the first body portion 210e. A longitudinal axis N₁ (rotation axis) of the rotation shaft 210g extends in a direction different from the central axis (axis N₂ illustrated in FIG. 18) of the shaft 23. For example, the longitudinal axis of the rotation shaft 210g extends in a direction perpendicular to the central axis of the shaft 23. The second body portion 210f is rotatable about the rotation shaft 210g and is fixed at a set position. The rotation shaft 210g moves the first body portion 210e on a plane intersecting the direction in which the probe body 201 and the jaw 210 face each other and orthogonal to the rotation shaft 210g. In the fourth embodiment, the first body portion 210e, the second body portion 210f, and the rotation shaft 210g constitute an adjustment mechanism.

FIG. 19 is a view for describing an adjustment method according to the fourth embodiment of the disclosure. When the second body portion 210f is rotated about the rotation shaft 210g, the first body portion 210e is displaced in the direction of an arrow Q₄ with respect to the first body portion 210e. By this displacement, the relative position between the probe body 201 and the grasp portion 210a is adjusted, and the second body portion 210f is fixed at a position having a desired positional relationship.

In the fourth embodiment described above, the configuration related to the position adjustment of the probe body 201 and the grasp portion 210a is changed from that in the first embodiment described above, but the behavior of the treatment tool itself after the adjustment is not changed, in a manner that the same effect as that in the first embodiment can be obtained.

### Other Embodiments

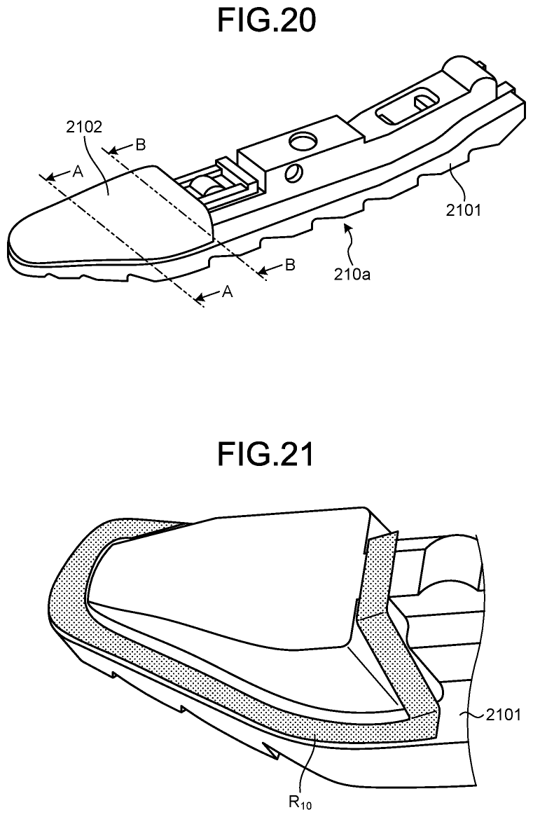

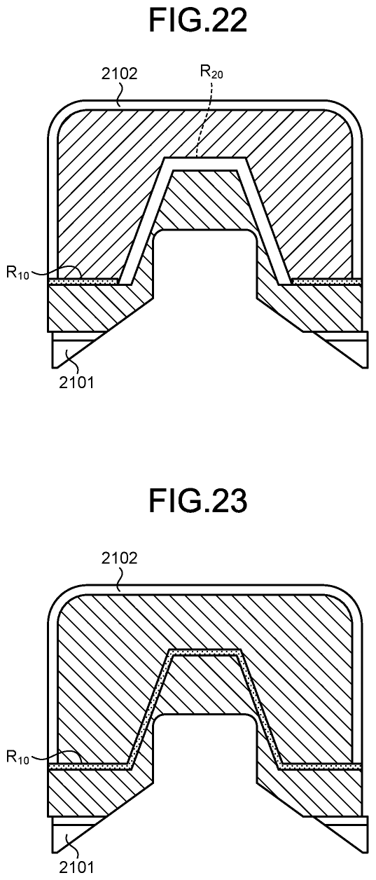

Next, Other embodiments will be described with reference to FIGS. 20 to 23. FIGS. 20 and 21 are views illustrating a configuration of a main portion of a treatment tool according to another embodiment. FIGS. 22 and 23 are cross-sectional views illustrating a configuration of the main portion of the treatment tool according to another embodiment. FIG. 22 is a view illustrating a cross section taken along line A-A in FIG. 20. FIG. 23 is a view illustrating a cross section taken along line B-B in FIG. 20. Note that the overall configuration of the treatment system according to the second embodiment is the same as that of the treatment system 1 described above except that the adjustment mechanism of the relative position between the probe body and the jaw is changed, and thus the description will be omitted. Hereinafter, a configuration different from that of the embodiments will be described.

The grasp portion 210a is attached to the body of the jaw 210, and includes an electrode portion 2101 that functions as an electrode that energizes a target region when the target region is grasped, and a cover 2102 attached to the electrode portion 2101. A bonding area R₁₀ to which the cover 2102 is bonded is set in the electrode portion 2101. The bonding area R₁₀ is set according to the outer edge of the cover 2102.

The outer edge of the cover 2102 corresponding to the bonding area R₁₀ is bonded to the electrode portion 2101 on the surface on the side facing the electrode portion 2101. On the other hand, the cover 2102 forms a space with the electrode portion 2101 inside the bonding area R₁₀. Specifically, the electrode portion 2101 and the cover 2102 form a space R₂₀ illustrated in FIG. 22 inside. Therefore, the peripheral surfaces of the electrode portion 2101 and the cover 2102 are in close contact with each other to hermetically seal the inside, and the space R₂₀ is formed inside. At this time, the bonding surface of the cover 2102 of the electrode portion 2101 and the forming surface of the space R₂₀ form an angle of 90° or more.

In the other embodiment described above, the electrode portion 2101, the cover 2102, and the outer peripheral surface are in close contact with each other, in a manner that the liquid is prevented from entering the cover 2102, and the space R₂₀ is formed inside, in a manner that the heat transferred to the electrode portion 2101 can be made difficult to be transferred to the cover 2102.

Furthermore, the jaw 210 body may be provided with a cover formed of a porous body. A plurality of independent spaces (air pools) are formed inside the cover. By attaching this cover to the jaw 210, heat transfer from the jaw 210 body to the cover can be suppressed. This cover (or jaw body and cover) is formed using foam molding or a 3D printer.

Although the embodiments for carrying out the disclosure have been described so far, the disclosure should not be limited only to the embodiments described above. The disclosure may include various embodiments and the like that are not described herein.

Note that, in the above embodiment, an example in which an ultrasonic wave is applied or a high-frequency power is supplied to the probe body has been described. However, the disclosure may be applied to a configuration in which a high-frequency power is not supplied and only an ultrasonic wave is applied, or may be applied to a treatment tool for grasping in which a high frequency is not supplied and an ultrasonic wave is not applied.

The treatment tool according to the disclosure described above is useful for adjusting the positions at which the graspers face each other with high accuracy.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment tool comprising:
a first grasper;
a second grasper that is rotatably provided with respect to the first grasper, the second grasper being configured to grasp a target region together with the first grasper by approaching the first grasper;
a shaft configured to rotate the second grasper with respect to the first grasper; and
an adjustment mechanism configured to adjust a trajectory along which the second grasper passes when the second grasper rotates about the shaft and approaches the first grasper,
the adjustment mechanism including a protrusion provided according to a predicted length to be required due to variations in components.

2. The treatment tool according to claim 1, further comprising:
a first gripped portion that is connected to a proximal end side of the first grasper; and
a second gripped portion that is connected to a proximal end side of the second grasper, wherein
the first gripped portion includes
a guide configured to sandwich a part of the second gripped portion and guide a rotational position of the second grasper continuous with the second gripped portion.

3. The treatment tool according to claim 2, wherein
the protrusion is provided on the second gripped portion.

4. The treatment tool according to claim 1, wherein
the adjustment mechanism is configured to move the second grasper with respect to the first grasper in a direction parallel to a longitudinal direction of the second grasper.

5. The treatment tool according to claim 2, wherein
the protrusion is provided on the guide.

6. The treatment tool according to claim 1, wherein
the first grasper is configured to vibrate by an ultrasonic wave.

7. The treatment tool according to claim 1, wherein
the first grasper and the second grasper are electrodes through which a high-frequency current flows.

8. A treatment tool comprising:
a first grasper;
a second grasper that is rotatably provided with respect to the first grasper, the second grasper being configured to grasp a target region together with the first grasper by approaching the first grasper;
a shaft configured to rotate the second grasper with respect to the first grasper;
a first gripped portion that is connected to a proximal end side of the first grasper; and
a second gripped portion that is connected to a proximal end side of the second grasper,
the treatment tool being configured such that a relative position between a first grasper and a second grasper is adjusted when the second grasper and the second gripped portion are rotatably coupled.

9. A method of adjusting a treatment tool, the method comprising:
checking a shift between a first grasper and a second grasper provided rotatably with respect to the first grasper in a state where the first grasper and the second grasper are aligned; and
adjusting a relative position between the first grasper and the second grasper.

10. The method of adjusting a treatment tool according to claim 9, wherein
the treatment tool includes
a first gripped portion that is connected to a proximal end side of the first grasper, and
a second gripped portion that is connected to a proximal end side of the second grasper,
the first gripped portion includes
a guide configured to sandwich a part of the second gripped portion and guide a rotational position of the second grasper continuous with the second gripped portion, and
the first gripped portion or the second gripped portion is provided with a protrusion to perform the adjustment.

11. The method of adjusting a treatment tool according to claim 10, wherein a protrusion amount of the protrusion is adjustable by a screw.

12. The method of adjusting a treatment tool according to claim 9, wherein
the treatment tool includes a second gripped portion that is connected to a proximal end side of the second grasper, and
a coupling between a convex portion provided in the second grasper and a hole provided in the second gripped portion is adjusted in a manner that the relative position between the first grasper and the second grasper is adjusted.

13. The method of adjusting a treatment tool according to claim 9, wherein
the treatment tool includes a second gripped portion that is connected to a proximal end side of the second grasper,
a coupling portion between the second grasper and the second gripped portion is provided with a rotation shaft configured to rotatably couple the second grasper and the second gripped portion, and
the second grasper rotates about the rotation shaft in a manner that the relative position between the first grasper and the second grasper is adjusted.

14. The method of adjusting a treatment tool according to claim 9, wherein
the treatment tool includes a shaft configured to rotatably couple the first grasper and the second grasper, and
a spacer is provided between the second grasper and the shaft in a manner that the relative position between the first grasper and the second grasper is adjusted.

15. The method of adjusting a treatment tool according to claim 9, wherein
the first grasper vibrates by an ultrasonic wave.

16. The method of adjusting a treatment tool according to claim 9, wherein
the first grasper and the second grasper are electrodes through which a high-frequency current flows.
